(19) United States
(12) Patent Application Publication
(10) Pub. No.: US 2017/0258334 A1
(43) Pub. Date: Sep. 14, 2017

(54) RECTAL PROBE SYSTEM FOR DETECTING, MONITORING, AND TREATING HEAT STROKE

(71) Applicant: Central Michigan University, Mount Pleasant, MI (US)

(72) Inventors: Kevin C. Miller, Mount Pleasant, MI (US); Rene R. Shingles, Mount Pleasant, MI (US)

(21) Appl. No.: 15/455,476

(22) Filed: Mar. 10, 2017

Related U.S. Application Data
(60) Provisional application No. 62/306,143, filed on Mar. 10, 2016.

(51) Int. Cl. A61B 5/01; A61F 7/00; A61B 5/00

(52) U.S. Cl. CPC A61B 5/01; A61B 5/6867; A61B 5/7282; A61B 5/746; A61B 5/0008; A61B 5/4848; A61B 5/4836; A61F 7/00; A61B 2503/10; A61F 2007/0096

(57) ABSTRACT

Systems and methods are described for detecting heat stroke and monitoring cooling treatment of heat stroke. The system includes a rectal temperature probe including a flexible length and an indicator positioned on the flexible length to indicate when the probe has been inserted to a target insertion depth. A controller is configured to receive a signal from the probe indicative of a sensed temperature and to determine whether heat stroke is likely based on the sensed temperature. When monitoring cooling treatment, a cooling rate is calculated based on periodically sensed temperatures and, based on the calculated cooling rate, the system indicates whether the heat stroke is being adequately treated or if the current treatment methods are insufficient.

### RELATED APPLICATIONS

This application claims the benefit of U.S. Provisional Patent Application No. 62/306,143, filed Mar. 10, 2016, entitled “RECTAL PROBE SYSTEM FOR DETECTING, MONITORING, AND TREATING HEAT STROKE,” the entire contents of which are incorporated herein by reference.

### BACKGROUND

Exertional heat stroke (EHS) is one of the leading causes of sudden death in athletes. EHS is diagnosed when body temperature exceeds 40° C. and the athlete displays signs or symptoms of central nervous system dysfunction. However, the signs and symptoms of EHS can vary considerably (e.g., confusion, irritability) and can mimic those of other serious conditions (e.g., concussions). Therefore, it is essential to obtain an accurate, valid measure of body core temperature (T_{core}) to confirm EHS diagnosis. By accurately diagnosing EHS and monitoring T_{core}, clinicians can implement proper treatment protocols (e.g., cold water immersion) and return-to-play criteria.

T_{core}, by definition, is the temperature of the hypothalamus. Given the difficulty of directly measuring brain temperature, other body sites for estimating T_{core} have been used and include the axilla, mouth, rectum, intestines (e.g., ingestible pills), esophagus, ear cannel, forehead, and pulmonary artery. While pulmonary artery temperature is considered the gold standard site for estimating T_{core}, it is prohibitively invasive and impractical to use in field settings. Many scientists prefer esophageal temperature (Tₑₛₒ) to estimate T_{core} because of its location (i.e., close to the heart), rapid response to acute temperature changes (e.g., exercise or body cooling), and correlation with pulmonary artery or aortic temperature. Since Tₑₛₒ is also invasive and impractical to use in the field, clinicians measure rectal temperature (T_{rec}) in EHS situations. T_{rec}, like Tₑₛₒ, provides valid estimates of T_{core} in exercising, hyperthermic humans and is practical in emergency situations.

### SUMMARY

Although prominent organizations, such as the National Athletic Trainers Association and the American College of Sport Medicine, recommend measuring rectal temperature (T_{rec}) if EHS is suspected, the precise procedures, materials, and techniques for doing so are not standardized. As described in detail below, the inventors have determined that the reliability, accuracy, and responsiveness of even rectal temperatures can vary significantly depending on the placement depth of a temperature probe. In some embodiments described below, the invention provides a rectal temperature probe with an indicator configured to confirm when the temperature probe has been inserted to an appropriate depth for accurate monitoring core temperature without inserting the probe to an unnecessarily intrusive depth.

In one embodiment, the invention provides a rectal temperature probe for detecting and monitoring heat stroke. The probe includes a flexible length for configured to sense a temperature when inserted rectally and an indicator positioned on the flexible length to indicate when the probe has been inserted to an appropriate depth. In some embodiments, the indicator includes a colored portion of the flexible length such that, when the probe is inserted to the appropriate depth, the entire colored portion of the flexible length is inserted rectally.

In some embodiments, the indicator includes a protrusion positioned on the flexible length such that the protrusion is placed immediately outside of the rectum when the probe is inserted to the appropriate depth. In some embodiments, the protrusion includes a spherical ball positioned axially along the flexible length at a fixed location.

In another embodiment, the invention provides a system for detecting heat stroke and for monitoring cooling treatment. The system includes a rectal temperature probe including a flexible length and an indicator positioned on the flexible length to indicate when the probe has been inserted to an appropriate depth. The system also includes a controller configured to receive a signal from the probe indicative of a sensed temperature and to determine whether heat stroke is likely based on the sensed temperature. The system is further configured to periodically sense the temperature and calculate a cooling rate. Based on the cooling rate, the system indicates whether the heat stroke is being adequately treated or if the current treatment methods are insufficient.

Other aspects of the invention will become apparent by consideration of the detailed description and accompanying drawings.

### BRIEF DESCRIPTION OF THE DRAWINGS

FIG. 1 is an overhead view of a rectal temperature probe for detecting and monitoring heat stroke according to one embodiment.

FIG. 2 is a block diagram of a control system for use with the rectal temperature probe of FIG. 1.

FIG. 3 is an elevation view of a user interface of the control system of FIG. 2

FIG. 4 is a flowchart of a method implemented by the control system of FIG. 2 to detect and monitor treatment of heat stroke using the rectal temperature probe of FIG. 1.

FIG. 5 is a graph comparing temperature readings measured during and after exercise using an esophageal temperature sensor and the rectal temperature probe of FIG. 1 inserted to a depth of 4 cm.

FIG. 6 is a graph comparing temperature readings measured during and after exercise using an esophageal temperature sensor and the rectal temperature probe of FIG. 1 inserted to a depth of 10 cm.

FIG. 7 is a graph comparing temperature readings measured during and after exercise using an esophageal temperature sensor and the rectal temperature probe of FIG. 1 inserted to a depth of 15 cm.

FIG. 8 is a graph of the statistical temperature bias demonstrated in FIGS. 5, 6, and 7 for the rectal temperature probe of FIG. 1 inserted at depths of 4 cm, 10 cm, and 15 cm, respectively.

FIG. 9 is a graph of the statistical temperature bias demonstrated during various measurement periods before, during, and after exercise.

FIG. 10 is a series of three graphs of Bland-Altman plots demonstrating the difference between the rectal temperature probe measurement and the esophageal temperature measurement for each of three rectal probe insertion depths.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

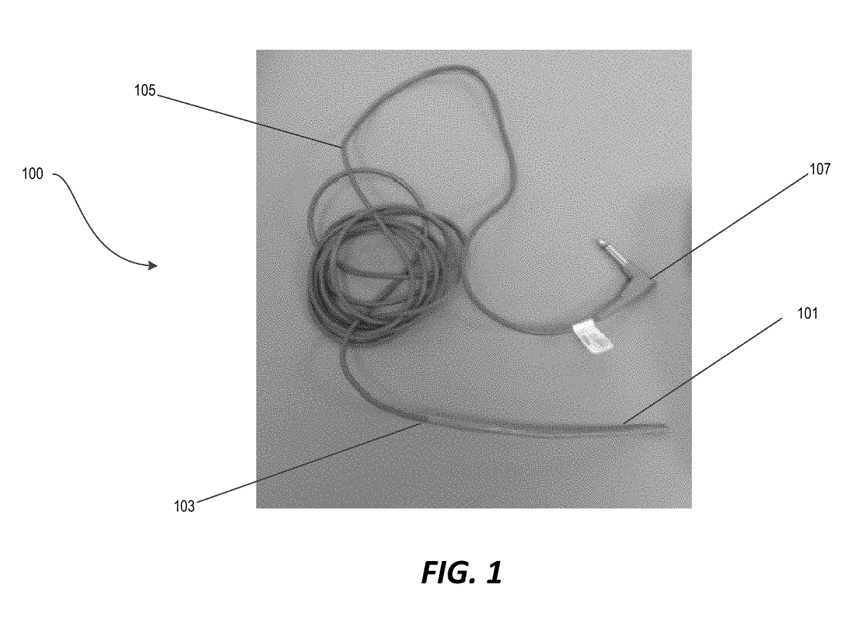

FIG. 1 illustrates a rectal temperature probe 100. The rectal temperature probe 100 includes a flexible length 101. The distal tip of the flexible length 101 of the rectal temperature probe 100 is inserted rectally to provide a temperature measurement. In the example of FIG. 1, the flexible length 101 is colored red from the distal tip until a position 103 located 15 cm from the distal tip of the flexible length 101. This colored portion of the flexible length provides an indication of an appropriate insertion depth from the rectal probe 100. The flexible length 101 of the rectal probe 100 is to be inserted rectally until the end of the colored portion 103 is no longer visible externally.

Although the example of FIG. 1 uses a colored portion of the flexible length 101 as an indicator of appropriate insertion depth, other indicators may be included in other implementation. For example, the rectal temperature probe 100 may be formed with a spherical projection fixedly positioned at location 103 on the flexible length 101. The spherical projection may be formed axially as an integral part of the flexible length 101 or, alternatively, may be a separate component that is otherwise fixedly coupled to the flexible length 101 at location 103. The spherical projection is sized with a diameter that is larger than the diameter of the flexible length (for example, a spherical diameter of ⅜″). In such embodiments, the flexible length 101 of the rectal probe 100 is to be inserted rectally until the spherical projection positioned at location 103 is located just outside of the rectum. Other projection-type indicators such as, for example, conical, cuboid, or linear projections can be similarly implemented in other rectal temperature probes.

The rectal temperature probe 100 includes a length of cable 105 connecting the flexible length 101 to an electrical coupling 107, such as, for example, the ¼″ jack coupling shown in the example of FIG. 1. In this example, the rectal temperature probe 100 has a total length of approximately 8-10 feet (including the flexible length 101 that is to be inserted and the length of cable 105), has a temperature range from −40 to 130 degrees Celsius, and is covered at least partially by a vinyl or Teflon sheathing.

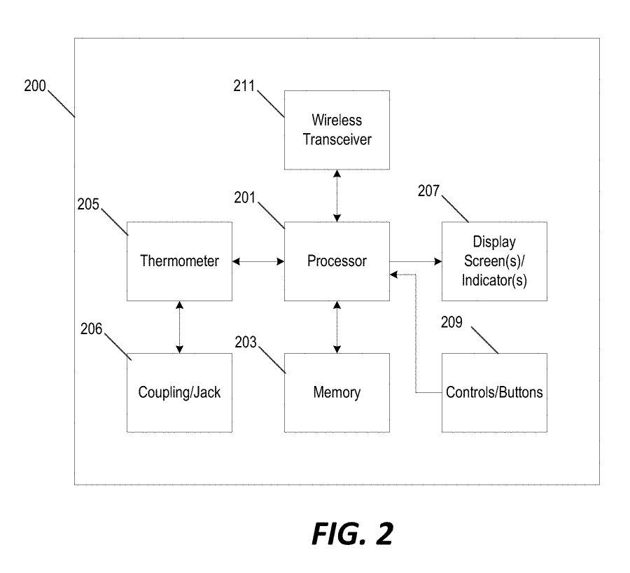

The electrical coupling 107 of the rectal temperature probe 100 is connected to a control system 200 such as illustrated in FIG. 2. The control system 200 in this example includes an electronic processor 201 that is communicatively coupled to a memory 203. The processor 201 accesses and executes instructions stored on a non-transitory computer-readable memory 203 to provide the functionality and operations such as described herein. The processor 201 is also coupled to a thermometer component 205. The thermometer component 205 receives a signal from the rectal temperature probe through a coupling/jack 206 on the control system housing. For example, in some implementations, the rectal temperature probe is designed such that its electrical resistance will change based on the temperature at the tip of the probe. In such implementations, the thermometer 205 applies a current to the probe through the coupling 206 and determines a sensed temperature based on the voltage of the probe.

The control system 200 also includes one or more display screens or other indicators 207 for outputting data or visual signals to a user. It also includes one or more controls or buttons 209 for receiving control inputs from the user. In some embodiments, the control system 200 also includes a wireless transceiver 211. The wireless transceiver can be used to transmit or receive data to remote systems such as a desktop computer, a tablet computer, or a remote computer server. In one particular example, the temperature probe 100 illustrated in FIG. 1 is inserted rectally in an athlete during exercise or competition. The control system 200 continuously or periodically monitors the core temperature of the athlete. The processor 201 may be configured such that, if the athlete's core temperature becomes indicative of potential heat stroke, the processor 201 causes the wireless transceiver 211 to send a signal or message to a computer device operated by a medical professional warning of the potential condition. When the warning message is received, the medical professional can take action to stop, limit, or monitor the athlete's current activities in order to prevent or treat the possible heat stroke as necessary.

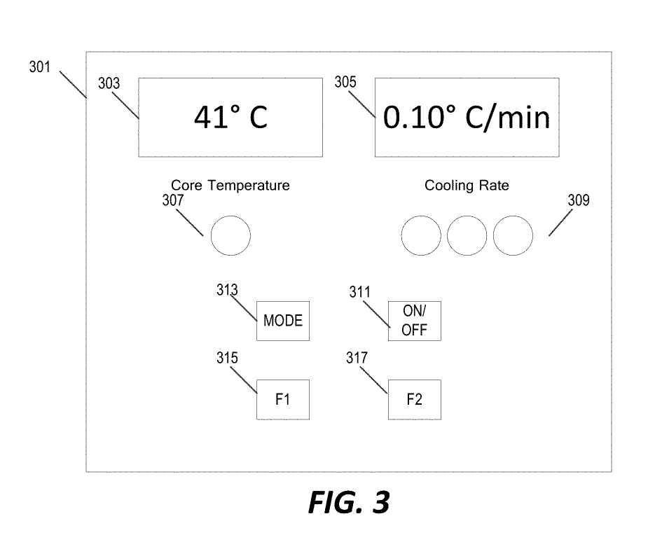

FIG. 3 illustrates one example of a user interface for use with the control system of FIG. 2. In this example, the user interface 301 is positioned on an exterior surface of a housing that contains the control system such as illustrated in FIG. 2. The user interface includes two display screen outputs: display screen 303 is configured to display the current temperature reading of the rectal temperature probe and display screen 305 is configured to display the current cooling rate as indicated by the temperature probe. As discussed in detail below, in some implementations, the cooling rate displayed on screen 305 is used to monitor the effectiveness of a treatment for heat stroke.

The user interface 301 of FIG. 3 also includes one or more indicators 307 positioned below the display screen 303 to provide a visual indication of a detected condition relating to the detected temperature. For example, as discussed in further detail below, the control system may be configured to activate the indicator 307 when the detected temperature exceeds a threshold indicative of possible heat stroke. In other examples, the indicator 307 may include a single multi-colored LED indicator and the controller may be configured to activate the indicator 307 in a first color (e.g., green) when no adverse condition is detected and in a second color (e.g., red) when potential heat stroke is detected. In still other examples, the controller may be configured to activate the indicator 307 in yet a third color (e.g., yellow) when the sensed temperature is determined to be indicative of possible heat exhaustion, but is not yet indicative of possible heat stroke. In yet other examples, the single indicator 307 illustrated in the example of FIG. 3 is replaced with a series of two or more indicators each configured to output a visual indication of the current detected condition (e.g., one indicator lit when the sensed temperature is normal and the other indicator is lit when the sensed temperature indicates potential heat stroke).

The user interface 301 of FIG. 3 similarly includes one or more indicators 309 positioned below the display screen. In the illustrated example, the three indicators each light in a different color (e.g., red, yellow, and green). The red indicator is lit when the calculated cooling rate indicates that the currently applied treatment of a detected heat stroke condition is not effective. The yellow indicator is lit when the calculated cooling rate indicates that the currently applied treatment of the detected heat stroke condition is effective, but not ideal. The green indicator is lit when the calculated cooling rate indicates that the currently applied treatment is ideal and effective.

Although the example of FIG. 3 illustrates two display screens 303, 305 each configured to display specific information, other display configurations are possible. For example, another user interface may include only a single display screen 303 displaying the current measured temperature. Alternatively, other implementations may have a single, more advanced display screen that is configured to display additional or alternative information including, in some cases, advanced menus and graphical outputs. Similarly, although the example of FIG. 3 shows one or more indicators 307, 309 positioned below the display screens 303, 305, in some other implementations, the display screens themselves may be configured to change color in order to indicate a condition. For example, display screen 305 may be configured to light red when the cooling rate is below a threshold indicative of effective treatment of heat stroke, to light yellow when the cooing rate is above the threshold indicative of effective treatment but below a second threshold indicative of ideal treatment, and to light green when the cooling rate is above the second threshold indicative of ideal treatment.

Lastly, the user interface 301 illustrated in FIG. 3 includes a series of buttons including an “on/off” button 311, a “mode” button 313, a first function (“F1”) button 315, and a second function (“F2”) button 317. However, in other implementations, the user interface may include more, fewer, or different buttons or controls for receiving control input commands from a user. For example, in some such implementations, the functionality and operations of the displays 303, 305, the indicators 307, 309, and the buttons 311, 313, 315, 317 are all provided by a single touch-sensitive display.

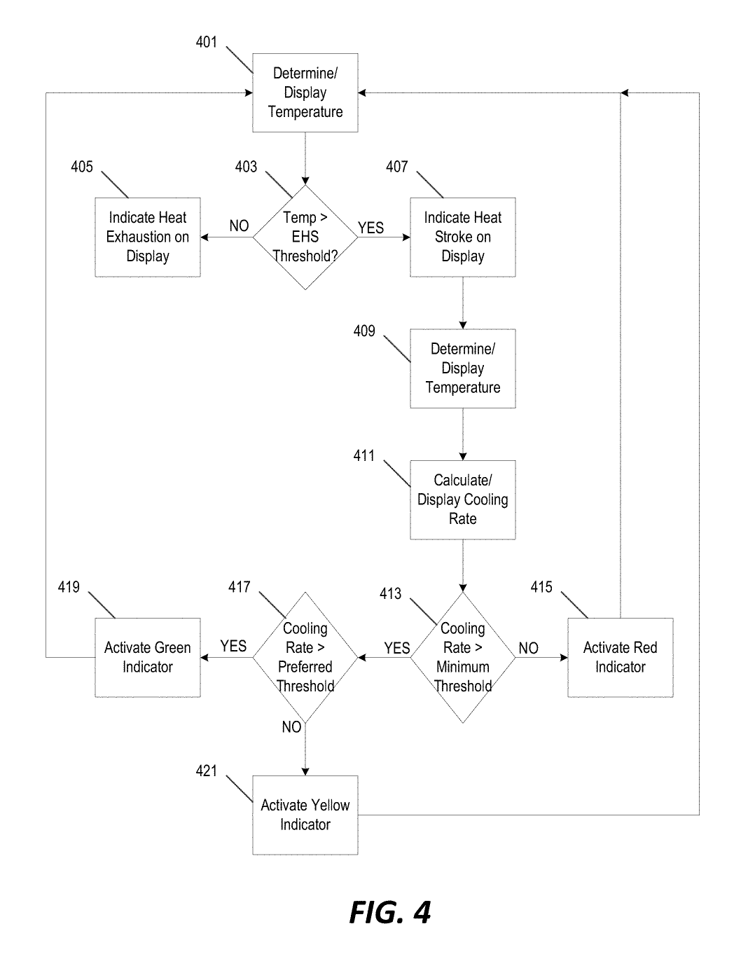

FIG. 4 illustrates a method for detecting and monitoring heat stroke and treatment of heat stroke using the rectal temperature probe of FIG. 1, the control system of FIG. 2, and the user interface of FIG. 3. This method may be continuously implemented periodically or as a loop when the temperature probe is rectally inserted in an athlete during exercise or competition. It may similarly be implemented in emergency situations after a person collapses—the rectal temperature probe may then be inserted after responsiveness and vital signs are assessed.

The control system determines a current temperature indicated by the output of the temperature probe 100 and displays the determined temperature on the display 303 (step 401). The determined temperature is then compared to a temperature threshold that is indicative of heat stroke (e.g., 40.5 degrees Celsius) (step 403). If the sensed temperature is below the threshold, the control system determines that the condition is not heat stroke and, in some implementations, may activate the indicator 307 in a way that indicates heat exhaustion (e.g., lit yellow) (step 405) (e.g., in implementations where the system is utilized in an emergency situation after the person has collapsed). However, if the sensed temperature is above the threshold, the system determines that heat stroke is likely and activates the indicator 307 accordingly (step 407).

When heat stroke is detected, treatment often includes removing extra layers of clothing/equipment, rapidly cooling the body in ice-water immersion, and monitoring core body temperature. During this treatment, the system continues to determine the sensed determine and updates the display (step 409). The system also uses the series of sensed temperatures to calculate a “cooling rate” that is then shown on the second display 305 (step 411). If the cooling rate is too slow, this may indicate that the current treatment is insufficient and is not effectively treating the heat stroke condition.

The control system 201 monitors the calculated cooling rate to evaluate the effectiveness of the treatment by using two different thresholds. If the cooling rate is below a “minimum” threshold (step 413), the system determines that the treatment is ineffective and activates the red indicator 309 (step 415). If the cooling rate is above the “minimum threshold,” the system then compares the cooling rate to a second “preferred” threshold (step 417). If the cooling rate is above the “preferred” threshold, the system determines that the heat stroke is being treated appropriately and the system activates the green indicator 309 (step 419) to indicate that the person appears to be recovering. If the cooling rate is above the “minimum” threshold and below the “preferred” threshold, the system determines that the treatment does appear to be working, but the person is not responding ideally, and the system activates the yellow indicator 309 (step 419) to indicate this intermediate treatment condition. In some implementations, the “minimum” threshold is set to 0.09 degree Celsius per minute and the “preferred” threshold is set to 0.15 degrees Celsius per minute.

In the example of FIG. 4, the system continues to monitor the temperature and cooling rate by looping back to step 401 until the temperature sensed by the rectal temperature probe indicates that heat stroke is no longer present (step 403) or until the system is turned off by the user.

As discussed above, the rectal temperature probe of FIG. 1 is configured to include an indicator mechanism to quickly inform the user when the probe has been inserted to an appropriate depth. This is because, although rectal temperature can provide a reliable approximation of core body temperature, the approximation becomes more accurate and reliable when the rectal probe is inserted to a specific depth—the temperature sensed by the rectal temperature probe of FIG. 1 can vary by as much as 0.84 degrees Celsius depending on the insertion depth. Thus, rectal insertion depth may impact diagnosis and treatment of exertional heat illnesses. To demonstrate this, a study was conducted to compare the sensed rectal temperature (T_{rec}) at three different insertion depths: 4 cm (T_{4 cm}), 10 cm (T_{10 cm}), and 15 cm (T_{15 cm}). The sensed temperatures were each compared to a corresponding temperature sensed by an esophageal probe (Tₑₛₒ).

Test subjects donned a heart rate monitor and an esophageal thermistor was inserted 42 cm into the esophagus via the nasal passage. This distance ensured the tip of the thermistor was below the tracheal bifurcation and near the level of the left ventricle. Subjects then self-inserted the rectal temperature probe. The rectal probe used for this test included three insulated thermocouples permanently affixed within a single protective casing so that T_{rec} could be measured at three different depths: 4 cm, 10 cm, and 15 cm. To prevent further movement of the probe during testing, it was secured to the subjects lower back with non-adhesive tape.

Subjects entered an environmental chamber and stood on a treadmill for 10 minutes to acclimate to the heat (40.3° C., 26.7% relative humidity). Following this rest period, subjects performed an incremental exercise protocol consisting of walking for 3 minutes at 3 mph and running at 90% of their age-predicted maximum heart rate for 2 min (0% incline). After each 5-minute bout, subjects stopped the treadmill and rested for 30 seconds. During this time, subjects palpated their anus to confirm the rectal thermistor was at the appropriate depth. T_{rec} was then recorded. Following this 30-second rest period, subjects resumed walking at 3 mph for the remainder of their 3-minute walking period. This walking/running/rest protocol continued until Tₑₛₒ reached 39.5° C. Tₑₛₒ was monitored continuously to determine when subjects reached 39.5° C.

Upon reaching a Tₑₛₒ of 39.5° C., subjects stopped the treadmill, checked the depth of the rectal thermistor, and had their T_{rec} recorded. They stepped off the treadmill, removed only their shoes, and entered a 1135.6 L capacity, non-circulating water tub (160.7 cm [L]×175.3 cm [W]×63.5 cm [H]). Subjects immersed themselves, up to the neck, for the duration of cooling. A stopwatch was started the moment each subject's foot touched the water. Subjects remained in the water bath until all temperatures read ≦38° C. The exact time to reduce all temperature sites to 38° C. was recorded so we could calculate cooling rates.

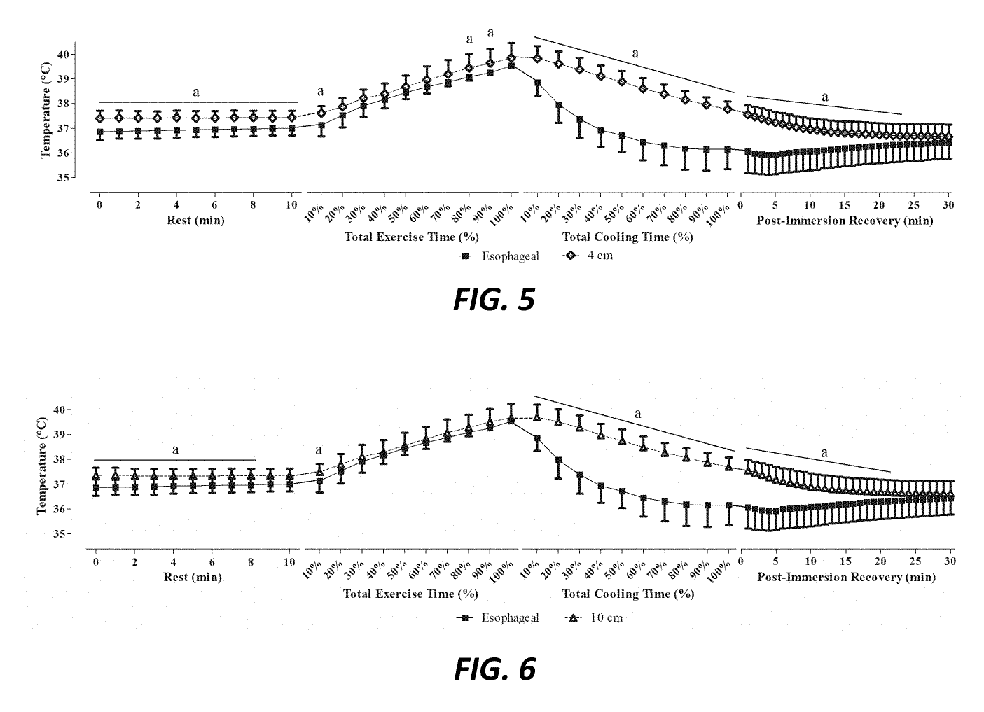

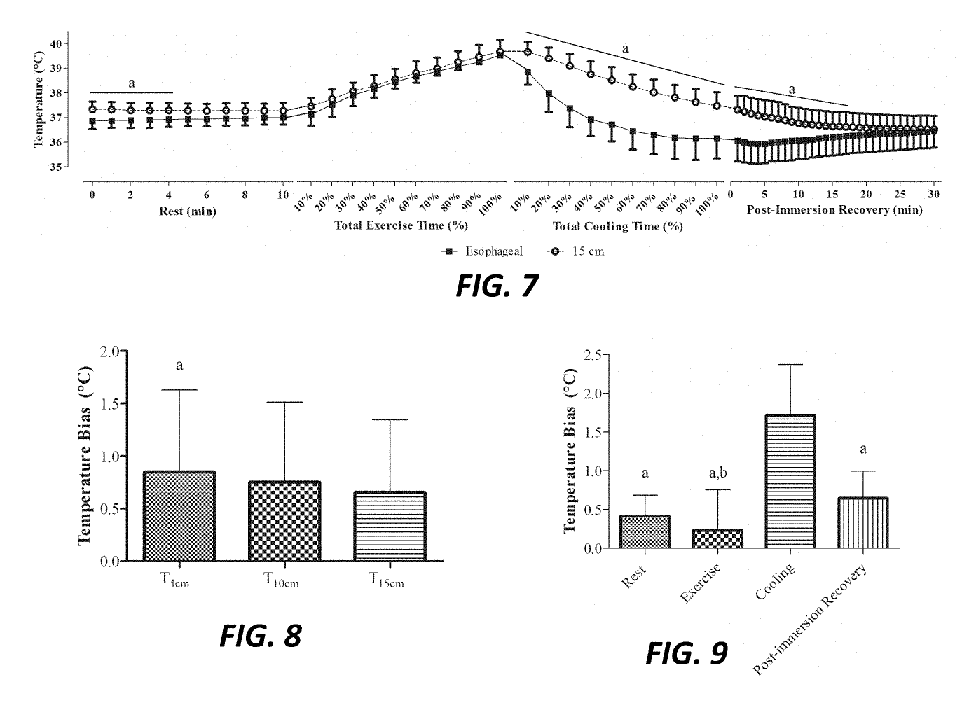

FIG. 5 illustrates the mean temperatures sensed by the rectal probe at the 4 cm depth as compared to the corresponding mean esophageal temperature readings. FIG. 6 illustrates the mean temperatures sensed by the rectal probe at the 10 cm depth as compared to the corresponding mean esophageal temperature readings. FIG. 7 illustrates the mean temperatures sensed by the rectal probe at the 15 cm depth as compared to the corresponding mean esophageal temperature readings. FIGS. 5-7 also illustrate the standard deviation of each sensor reading. In FIGS. 5-7, data points (or groups of data points) where the rectal temperature is statistically different than the esophageal temperature (i.e., P<0.05) are identified with an “a” notation.

FIG. 8 illustrates the overall bias for rectal depth and FIG. 9 illustrates the overall bias for experimental period. In FIG. 8, the “a” notation indicates that the temperature bias for T_{4 cm} is significantly higher than T_{15 cm} (P<0.05). In FIG. 9, the “a” notation indicates that the temperature bias during the rest, exercise, and post-immersion recovery period is significantly less than the temperature bias during the cooling period (P<0.05). The “b” notation in FIG. 9 indicates that the temperature bias during the exercise phase is significantly less than the temperature bias during the post-immersion recovery period (P<0.05).

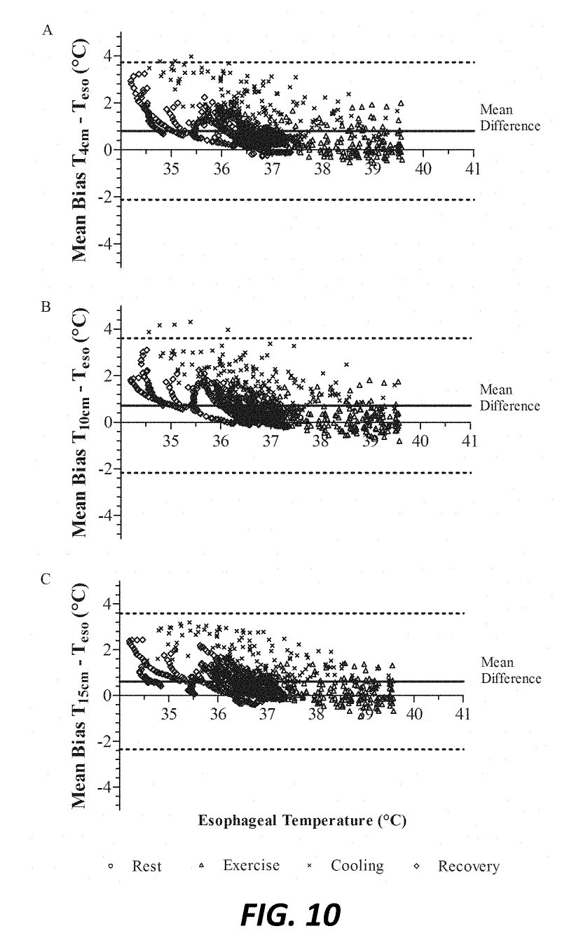

FIG. 10 provides the Bland-Altman plots indicating temperature bias between esophageal temperature and rectal temperature at 4 cm (Graph A), 10 cm (Graph B), and 15 cm (Graph C) from the anal sphincter. The dashed lines on each graph represent the 95% limits of agreement. The bold solid line in each graph indicates the mean difference.

The data illustrated in FIGS. 5-10 demonstrate that rectal depth affected bias. In particular, bias at T_{4 cm} was significantly higher than T_{15 cm}, but not T_{10 cm}.

Thus, the invention provides, among other things, a rectal temperature probe with a fixed indicator for appropriate insertion depth for appropriate detection and monitoring of heat stroke and treatment of heat stroke. Various features and advantages of the invention are set forth in the following claims.

What is claimed is:

1. A body heat monitoring system, the system comprising:
a rectal temperature probe including
a flexible length configured to sense a temperature when inserted rectally, and
an indicator positioned on the flexible length indicative of a target insertion depth for detecting and monitoring heat stroke.

2. The body heat monitoring system of claim 1, wherein the flexible length of the rectal temperature probe is configured to remain inserted rectally in an athlete during athletic performance.

3. The body heat monitoring system of claim 1, wherein the indicator includes a colored portion of the flexible length such that, when the flexible length is inserted to the target insertion depth, the entire colored portion of the flexible length is inserted rectally.

4. The body heat monitoring system of claim 1, wherein the indicator includes a protrusion positioned on the flexible length such that the protrusion is located immediately outside a rectum of a user when the flexible length is inserted to the target insertion depth.

5. The body heat monitoring system of claim 4, wherein the protrusion includes a substantially spherical protrusion positioned axially along the flexible length at a fixed location.

6. The body heat monitoring system of claim 1, wherein the indicator is positioned on the flexible length at a location indicative of a target insertion depth of 15 centimeters.

7. The body heat monitoring system of claim 1, further comprising a controller configured to:
receive a signal from the rectal temperature probe indicative of a sensed temperature, and
determine whether a heat stroke condition is likely present based on the sensed temperature.

8. The body heat monitoring system of claim 7, wherein the controller is further configured to generate an alert signal in response to determining that the heat stroke condition is likely present.

9. The body heat monitoring system of claim 8, further comprising a wireless transmitter, and wherein the controller is configured to generate the alert signal by causing the wireless transmitter to transmit the alert signal to a remote computer system where an indication of the heat stroke condition.

10. The body heat monitoring system of claim 9, further comprising the remote computer system, wherein the remote computer system configured to
receive signals indicative of sensed temperatures from a plurality of rectal temperature probes, each inserted rectally into a different one of a plurality of athletes, and
output on a display, for each individual athlete of the plurality of athletes, an indication of the sensed temperature and an indication of whether the alert signal has been generated indicative of the heat stroke condition.

11. The body heat monitoring system of claim 1, further comprising a controller configured to
periodically determine a sensed temperature of an individual subject based on a signal received from the rectal temperature probe,
calculate a cooling rate based on the periodically determined sensed temperature,
determine, based on the calculated cooling rate, whether a current treatment of a heat stroke condition is adequate, and
generating an ineffective treatment signal in response to determining, based on the calculated cooling rate, that the current treatment of the heat stroke condition is not adequate.

12. The body heat monitoring system of claim 11, wherein the controller is configured to determine, based on the calculated cooling rate, whether the current treatment of the heat stroke condition is adequate by comparing the calculated cooling rate to target treatment cooling rate threshold.

13. The body heat monitoring system of claim 1, further comprising:
an ideal treatment condition indicator;
a mid-level treatment condition indicator;
an ineffective treatment condition indicator; and
a controller configured to
periodically determine a sensed temperature of an individual subject based on a signal received from the rectal temperature probe,
calculate a cooling rate based on the periodically determined sensed temperature,
activate the ineffective treatment condition indicator in response to determining that the calculated cooling rate is less than a first cooling rate threshold,
activate the mid-level treatment condition indicator in response to determining that the calculated cooling rate is greater than the first cooling rate threshold and less than a second cooling rate threshold, wherein the second cooling rate threshold is greater than the first cooling rate threshold, and
activate the ideal treatment condition indicator in response to determining that the calculated cooling rate is greater than the second cooling rate threshold.

14. The body heat monitoring system of claim 1, wherein the ideal treatment condition indicator includes a green LED, wherein the mid-level treatment condition indicator includes a yellow LED, and wherein the ineffective treatment condition indicator includes a red LED.

15. A method of detecting heat stroke in a subject while the subject is engaged in a physical activity, the method comprising:
inserting a rectal temperature probe rectally into the subject to a target insertion depth, the rectal temperature probe including
a flexible length configured to remain inserted rectally while the individual engages in the physical activity and to sense a body temperature of the individual while inserted rectally, and
an indicator positioned on the flexible length indicative of whether the flexible length is inserted to the target insertion depth;
receiving, by an electronic controller, a signal from the rectal temperature probe indicative of the sensed body temperature of the subject;
detecting, based on the received signal indicative of the sensed body temperature, a potential heat stroke condition; and
outputting on a display an indication that the potential heat stroke condition has been detected in response to detecting the potential heat stroke condition.

16. A method of monitoring treatment of heat stroke, the method comprising:
inserting a rectal temperature probe rectally into a subject to a target insertion depth, the rectal temperature probe including
a flexible length configured to remain inserted rectally while the individual engages in the physical activity and to sense a body temperature of the individual while inserted rectally, and
an indicator positioned on the flexible length indicative of whether the flexible length is inserted to the target insertion depth;
receiving, by an electronic controller, a signal from the rectal temperature probe indicative of the sensed body temperature;
calculating, by the electronic controller, a cooling rate based on the signal received from the rectal temperature probe while a first heat stroke treatment is applied to the subject;
determining, by the electronic controller, whether the first heat stroke treatment is adequate based on the calculated cooling rate; and
outputting on a display an indication of whether the first heat stroke treatment is adequate in response to determining whether the first heat stroke treatment is adequate.
